(19) United States
(12) Patent Application Publication
(10) Pub. No.: US 2025/0387613 A1
(43) Pub. Date: Dec. 25, 2025

(54) MEMBRANE CROSSING RIGHT CARDIAC ASSISTING DEVICE

(71) Applicant: BRIGHTFLOW, Orléans (FR)

(72) Inventors: Océane-Aurore HELLUY, Orléans (FR); Aurélien NICOL, Orléans (FR); Anudeep SAMI, Orléans (FR)

(73) Assignee: BRIGHTFLOW, Orléans (FR)

(21) Appl. No.: 18/880,408

(22) PCT Filed: Jun. 30, 2023
(86) PCT No.: PCT/EP2023/068008

§ 371 Date: Dec. 31, 2024

(30) Foreign Application Priority Data
Jul. 1, 2022 (EP) 22305989.0

(51) Int. Cl. A61M 60/165; A61M 60/216; A61M 60/585; A61M 60/802; A61M 60/871

(52) U.S. Cl. CPC A61M 60/165; A61M 60/216; A61M 60/585; A61M 60/802; A61M 60/871

(57) ABSTRACT

A right cardiac assisting device to be percutaneously implanted inside a patient's heart, including an inlet, an outlet, a pump connecting the inlet to the outlet, and being designed to be located inside the right atrium or vena cava of the patient. The assisting device includes at least one support element configured to be secured to a first membrane of the patient's heart by passing through the first membrane, the first membrane separating the right atrium from the pulmonary artery of the patient's by passing through it. The support element tightly cooperates with the outlet to immobilize the pump, the inlet and the outlet inside the patient's heart and enable the patient's blood flow to be driven from the inlet to the outlet through the first membrane.

### FIELD OF INVENTION

The present invention relates to a percutaneous implantable heart assist device.

### BACKGROUND OF INVENTION

Heart failure remains a major health problem, with an estimated prevalence of 1-2% in the adult population of developed countries increasing to 10% from the age of 70 years.

Nowadays most cardiac assist devices are implanted in the left ventricle as most of the past years research was oriented towards it. The left ventricle has been considered, for a very long time, as the only active part of the heart, as it is bigger and more powerful than the right ventricle. As a result, the right ventricle has been considered, up to recently, as a passive reservoir and was not considered significant enough to be included in the heart assist devices research. Therefore, the few assist devices already existing for the right side of the heart are all temporary and they are mostly outside the heart or the body, requiring immobility of patients.

Most patients suffering from right ventricle dysfunction actually suffer from bi-ventricular dysfunctions, as most of the right ventricle dysfunctions are a long-term consequence from left ventricle dysfunctions. Although they affect only a small number of heart failure patients, bi-ventricular dysfunctions are a major therapeutic challenge due to its appalling prognosis. Thus, today, the reference treatment for irreversible bi-ventricular dysfunctions remains heart transplantation. However, the criteria for eligibility for the transplant (selection of candidates) and the shortage of grafts make this therapy available for only a few selected patients. In addition, the waiting lists are very long, leading thus to long waiting times, which incompatible with the precarious health of some candidates.

One way of overcoming this lack of grafts, some systems have been developed based on the combination of Left Ventricular Assis Device (LVAD) and Right Ventricular Assis Device (RVAD) leaving in place the native heart and assisting the two ventricles by two external chambers. Although it is the most usable device in clinical practice, it remains subject to a major risk of complication (infectious, thromboembolic) and the complexity of the installation will increase the mortality risk during surgery.

As an alternative treatment, the development of total artificial hearts has gradually emerged, with the issue of allowing a return home under the cover of a good quality of life. However, there are many drawbacks that limit their development, including ergonomic limitations, heavy surgery, and the instantaneous death of the patient in the event of a pump stops.

In addition, most of those patients having bi-ventricular dysfunctions are aged patients and have severe comorbidities and/or diseases, and in this case, no reasonable therapeutic solution is available, as heavy open-heart surgery is excluded.

Despite these engineering difficulties, some actors have described some clinical cases and small series using two implanted LVADs for bi-ventricular failures. Even if the idea seems attractive, the combination of two LVADs potentially exposes to a double risk of complications (infectious, surgical . . . ) and by the limiting anatomical and physiological constraints of the right ventricle (small size and particular shape of the cavity, thinner walls and low pressure and low systemic resistance than the left ventricle). Finally, the use of LVADs devices outside of their official indication does not appear to be the solution that will satisfy the market.

In the absence of a satisfying solution to treat terminal bi-ventricular dysfunctions, the future directions include the miniaturization of LVADs allowing their implantation by percutaneous or mini-invasive access and the development of a right cardiac assistance fully implantable percutaneously to limit the surgical and infectious risk could be the solution to many patients without therapeutic project.

The development of a hybrid miniaturized RVAD used as a destination therapy combinate or not with LVAD, implanted without heavy surgery is the solution aimed at to increase the number of patients to be treated, especially patients who are not eligible for heart transplant. A further benefit is to avoid a re-intervention in case of complication or pump malfunction and the possibility to replace it percutaneously, especially in frail elderly patients. However, delivering an assist device percutaneously inside the right ventricle presents some issues like the sizing of its constitutive elements, and the positioning and securing of said elements inside the patient's heart.

Regarding human anatomy, the best location for the assist device is inside the ventricle of the heart. This way the assist device can support the superior and inferior vena cava as the inlet of the assist device would be situated in the common volume of both the inferior and the superior vena cava. Due to the uncertain anatomic situation inside the right ventricle (strong trabeculation, for example) it is preferable to place the assist device, and particularly the pump, inside the right atrium or inside the vena cava.

The technical problem to be solved by the present invention is thus to propose an assist device which can be safely implanted inside the ventricle of the human heart, without damaging neither the device and nor the patient's heart.

### SUMMARY

The present invention aims at solving this problem and thus relates to a right cardiac assisting device configured to be percutaneously implanted inside a patient's heart, said assisting device comprising:
an inlet,
an outlet conduct,
a rotary pump comprising a pump body surrounding a rotor, said rotary pump connecting the inlet to the outlet conduct, and being designed to be located inside the right atrium or vena cava of the patient,
   wherein
the assisting device further comprises at least one support element, the at least one support element is configured to be secured to a first membrane of the patient's heart by passing through said first membrane, the first membrane separating the right atrium or the superior vena cava from the pulmonary artery of the patient's heart by passing through said first membrane,
the at least one support element is configured to tightly cooperate with the outlet conduct in order to immobilize the pump, the inlet and the outlet conducts inside the patient's heart or vena cava and enable the patient's blood flow to be driven from the inlet to the outlet conduct through the first membrane of the patient's heart.

Thus, this solution achieves the above objective. In particular, it allows the obtaining of a functional assist device which is designed to be safely percutaneously implanted inside the patient's right heart without damaging neither the patient's heart nor the device itself.

The device according to the invention may include one or more of the following characteristics, taken in isolation from one another or in combination with one another:
the assisting device may further comprise a second support element configured to be secured to a second membrane of the patient's heart by passing through said second membrane, said second support element being configured to cooperate directly or indirectly with the pump body,
the first membrane may be the wall of the superior or inferior vena cava,
the second membrane may be the membrane separating the left atrium from the right atrium of the patient's heart,
the pump may be configured to be anchored to the patient's heart at one of the extremities of the pump body,
the rotor of the pump may be surrounded by one extremity of the pump body, the pump being configured to be anchored to the patient's heart at the extremity of the pump body surrounding the rotor,
the pump may be configured to be anchored to the patient's heart directly by the extremity of the pump body surrounding the rotor,
the pump may be configured to be anchored to the patient's heart by means of a connection element connecting the extremity of the pump body surrounding the rotor to the patient's heart,
the device may display a general Y shape or T shape,
each support elements may be deployable from a retracted configuration to an expanded configuration, the retracted configuration enabling each support element to be safely introduced through the first or second membrane of the patient's heart, and the expanded configuration enabling each support element to stay in place inside the first or second membrane,
each support element may comprise two expandable flanges, the expanded configuration of each support element enabling the pinching of the first or second membrane between the two flanges,
the first expandable flange may extend from the first extremity of the support element and the second expandable flange extends from the second extremity of the support element, the first expandable flange being configured to be located on a first side of the first or second membrane and the second expandable flange being configured to be located on a second side of the first or second membrane,
the rotor of the rotary pump may be part of a motor designed to be pushed inside the pump body in order to snap the motor and the pump body together,
the pump body may comprise a compression chamber configured to surround an impeller connected to the rotor.

A further object of the present invention is about a right assisting kit comprising:
a right assisting device according to any one of the preceding claims,
a control unit for controlling the rotary pump,
a power supply for supplying power to the rotary pump.

Finally, the present invention has also for object an implantation method for a right cardiac assisting device according to any one of the preceding claims, wherein the method includes following steps:
transpiercing the first and second membrane,
securing the first support element inside the first membrane,
introducing, the pump body and the outlet conduct through the first support element inside the right atrium of the patient's heart,
releasing the pump body and the outlet conduct inside the right atrium,
securing the outlet conduct (14) to the first support element,
locking the motor inside the pump body.

### BRIEF DESCRIPTION OF THE DRAWINGS

FIG. 1 is a schematic longitudinal section view of a patient's heart;

FIG. 2A is the same schematic view as FIG. 1, further displaying two holes created in the first and second membranes according to the present invention;

FIG. 2B is the same schematic view as FIG. 2A, in which the first and second support elements according to the present invention, have been added to the heart;

FIG. 2C is the same schematic view as FIG. 2B, in which the pump has been added to the support elements according to the present element;

FIGS. 3A and 3B are two schematic transparent perspective views of a patient's heart with an implanted device according to the present invention;

FIG. 4 is a perspective view of a pump body according to the present invention;

FIG. 5A is a perspective view of a motor according to the present invention facing the pump body;

FIG. 5B is a perspective view of the motor and the pump body according to the present invention, snapped together;

FIG. 6 is another schematic longitudinal section view of a patient's heart with an implanted device according to an alternative embodiment of FIG. 2C;

FIG. 7 is a perspective view of a support element according to the present invention,
FIG. 8 is a detailed perspective view of FIG. 7,
FIG. 9A and 9B are two perspective views of a support element according to the present invention cooperating with a membrane of the patient's heart.

### DETAILED DESCRIPTION

### Assist Device

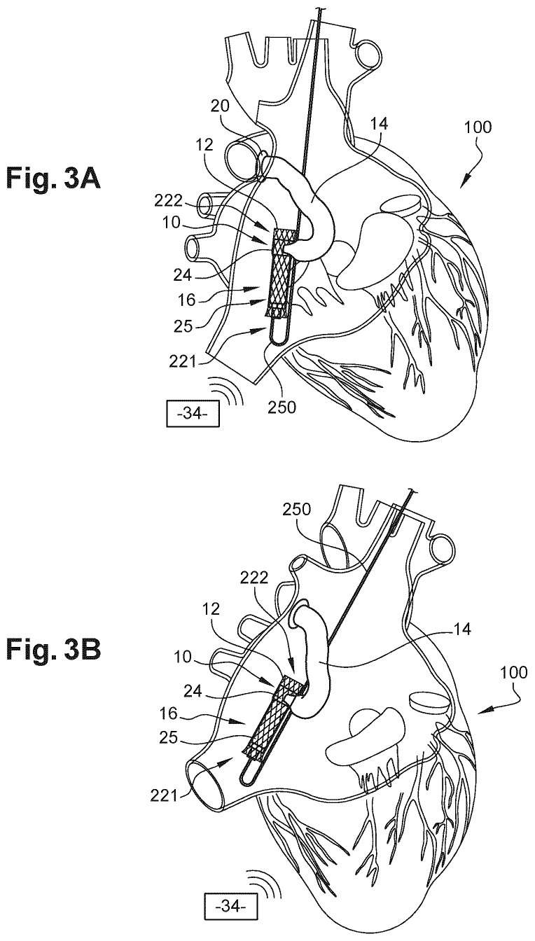

As can be seen on FIGS. 2A, 2B and 2C or on FIGS. 3A and 3B, the present invention is about a right cardiac assisting device 10 configured to be percutaneously implanted inside a patient's heart 100 or inside the vena cava 101

Therefore, said assisting device 10 comprises a series of independent and autonomous functional elements:
an inlet 12,
an outlet conduct 14,
a rotary pump 16 connecting the inlet 12 to the outlet conduct 14,
at least one support element 20 (distal or proximal anchoring).

In some alternative embodiment, the assisting device 10 comprises, beneath the inlet 12, the outlet conduct 14 and the rotary pump 16 connecting the inlet 12 to the outlet conduct 14:
a first support element 20 (distal anchoring),
and a second support element 18 (proximal anchoring).

To maintain the assisting device 10 in place, the device 10 needs to be anchored inside the patient's heart 100, at least on one of its extremities. This anchoring is achieved my means of the support elements 18, 20. More particularly, the pump 16 is configured to be anchored to the patient's heart 100 at one of the extremities of the pump body 22.

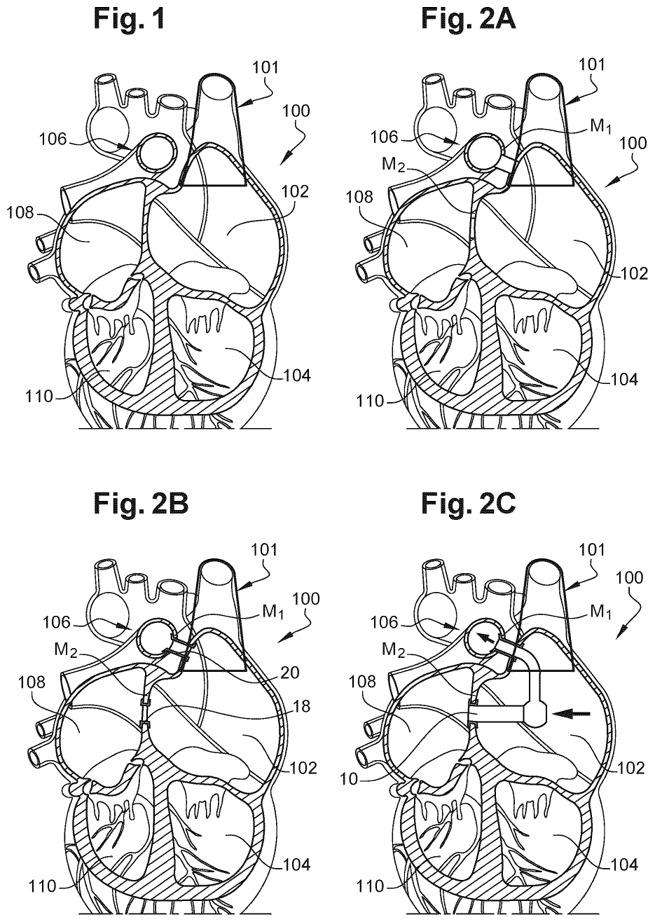

Classically, as can be seen on FIG. 1, the oxygen-poor blood flow coming from a patient's body is led to the patient's heart 100 by the inferior/superior vena cava 101 and enters the patient's heart 100 through the right atrium 102. The oxygen-poor blood flow then crosses the tricuspid valve and enters the right ventricle 104. The oxygen-poor blood flow then leaves the right ventricle 104 by crossing the pulmonary valve and flows towards the lungs through the pulmonary artery 106. The blood flow is oxygenated in the lungs and is fled once again towards the heart 100 by the pulmonary veins and reinters the heart 100 through the mitral valve inside the left atrium 108 and then into the left ventricle 110. The oxygen-rich blood flow then crosses the aortic valve and leaves the heart 100 through the aorta towards the patient's body. For a purpose of simplification, in the present invention, it is considered that the vena cava 101 is part of the human heart 100, in a broad interpretation of the human heart 100.

More precisely, the rotary pump 16 is designed to be located inside the right atrium 102 of the patient's heart 100. In some alternative embodiments (not represented), the rotary pump 16 is designed to be located inside the superior or inside the inferior vena cava 101.

The at least one support element 20 (or first support element 20 in the alternative embodiment with two support elements 18, 20) is configured to be secured to a first membrane M₁ separating the right atrium 102 from the pulmonary artery 106 of the patient's heart 100. The first membrane M₁ can also separate the superior vena cava 101 from the pulmonary artery 106 of the patient's heart 100.

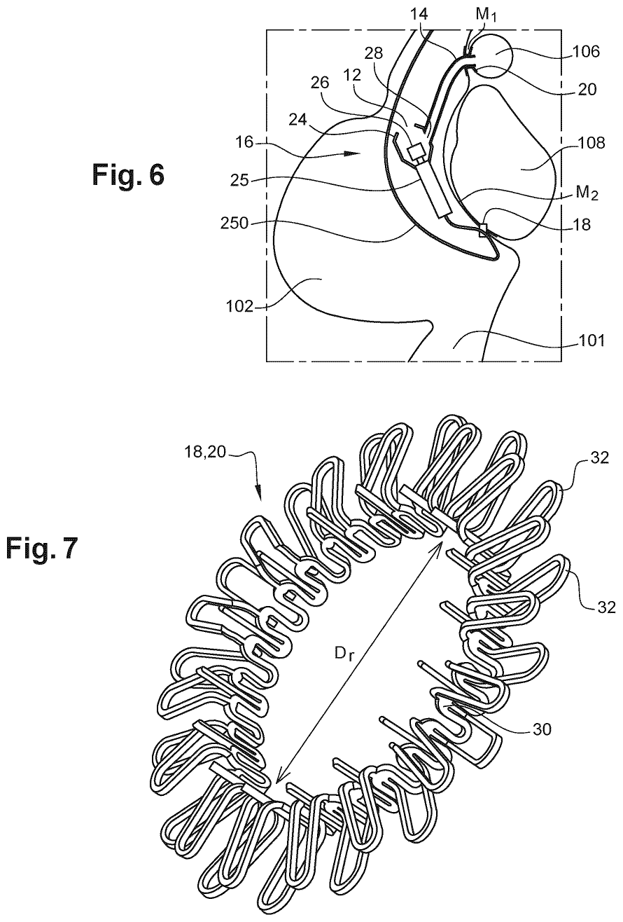

In the embodiments comprising two support elements 18, 20, the second support element 18 is configured to be secured to a second membrane M₂. This second membrane M₂ is preferably separating the left atrium 108 from the right atrium 102, as can be seen on FIGS. 2c and 6. In some alternative embodiment comprising two support elements 18, 20 (not represented), the second membrane M₂ can be the wall of the superior (or inferior) vena cava 101 or other structures of the right atrium 102.

In the present specification, the term “membrane” refers to a physiological area displaying the general shape of a membrane and which may include several anatomical structures, in coherence with the human heart's anatomy.

As mentioned above, the device 10 according to the present invention comprises a series of independent and autonomous functional elements 12, 14, 16, 18, 20 enabling significant length and diameter reductions and enabling a percutaneous implantation of it. This eases the implantation process significantly. Further, the specific patient's heart 100 anatomy with its very reduced space inside the right ventricle 104 does neither allow an accurate and precise positioning nor a satisfying stability of assist the device in case the functional elements 12, 14, 16, 18, 20 could not be inserted separately. More precisely, the right ventricle 104 is a complex three-dimensional structure, with a triangular shape in sagittal cut and a crescent-shaped cross-section. Its anatomy thus makes the percutaneous implantation of any support device difficult and in order to properly position the assist device 10 inside the right atrium 102, each support elements 18, 20 are designed to facilitate the delivery of the device 10 and correctly position the assist device 10 to accompany the blood flow. Also, the presence of at least three independent functional elements 16, 18, 20 enables to safely secure the device 10 to at least one anatomical parts of the patient's heart 100 without damaging the functioning of the device 10 and, in the embodiments comprising two support elements 18, 20, the presence of at least three independent functional elements 16, 18, 20 enables to safely secure the device 10 to two different and independent anatomical parts of the patient's heart 100. In the embodiments illustrated on FIGS. 2c and 6, those independent anatomical parts are, respectively the first membrane M₁ separating the right atrium 102 from the pulmonary artery 106, and the second membrane M₂ separating the left atrium 108 from the right atrium of the patient's heart 100.

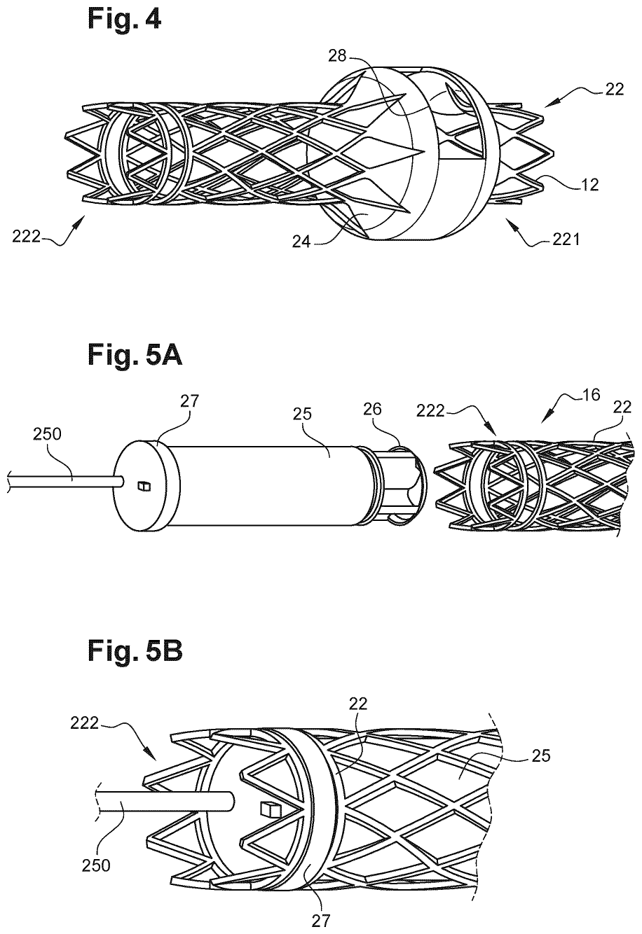

As can be seen on FIGS. 4 and 5A, the rotary pump 16 displays an elongated generally cylindrical shape extending along a revolution axis X. The rotary pump 16 thus comprises:
an elongated generally cylindrical pump body 22 (see FIG. 4) with a compression chamber 24,
a motor 25 with a rotor (not represented), and
a impeller 26 (see FIG. 5A).

As can be seen on FIG. 4, the pump body 22 has two extremities:
a first extremity 221 comprising the compression chamber 24,
a second extremity 222 configured to contain the motor 25 and, in some embodiments, further configured to cooperate with the second support element 18.

As can be seen on FIG. 4, the pump body 22, in some embodiments, the pump body 22 is at least partially, preferably completely, made of meshed material, for example nitinol. The used material might be nitinol, for example. This enables the pump body 22 to adopt a folded configuration or an unfolded configuration. The folded configuration ensures an easy implantation.

Once the rotary pump 16 is assembled, the motor 25 is located inside the second extremity 222 of the pump body 22 and the impeller 26 is located inside the compression chamber 24 of the pump body 22. The rotor of the motor 25 is configured to drive the impeller 26. The pump 16 is thus the merging of a motor and a impeller in order to generate a blood flow. The generated blood flow may be a continuous flow or a pulsatile flow, depending on the embodiments.

In order to enable the assembling of the pump 16, the motor 25 comprises a securing ring 27 (see FIGS. 5A and 5B). In the embodiment depicted on FIGS. 5A and 5B, the securing ring 27 displays a diameter slightly superior to the diameter of the motor 25 and can thus cooperates, by clamping, with a corresponding securing slot of the pump body 22. In some alternative embodiment (not represented), the securing ring 27 presents at least on securing groove. The securing ring 27 is therefore configured to cooperate with a complementary elongated securing element of the pump body 22 in order to lock the pump body 22 axially and radially with regards to the motor 25. Regardless of the embodiment, the securing ring 27 of the motor 25 and the pump body 22 functions as a ratchet mechanism to safely lock the motor 25 inside the pump body 22. In some embodiments the power supply of the motor 25 is assured by a motor cable 250. As can be seen on FIGS. 5A and 5B, the motor cable 250 and the motor 25 are connected at the second extremity 222 of the body pump 22.

The diameter of the motor 25 ranges from 6 to 12 mm and the length of the motor 25 ranges from 25 to 40 mm. The diameter of the impeller 26 ranges from 6 to 12 mm, preferably 9 mm and the length of the impeller 26 ranges from 4 to 10 mm, preferably 7 mm.

Once implanted and activated, the rotary pump 16 is designed to generate a blood flow rate ranging from 1 to 5 L/min (preferably 3 L/min) and a pressure ranging from 10 to 75 mmHg (preferably 20 mmHg). Once put in motion, the impeller 26 rpm ranges from 6 000 to 30 000, preferably ranging from 8 000 to 30 000.

In some embodiments, in order to avoid any kind of bloodstream reflux, the device 10 further comprises an anti-reflux system (not represented). This anti-reflux system might be integrated in the rotary pump 16 or might be, in some other embodiment, a one-way valve situated in the outlet conduct 14.

The compression chamber 24 comprises the device inlet 12 and a chamber outlet 28. The device inlet 12 is an axial opening situated at the first extremity 221 of the pump body 22. The chamber outlet 28 is a radial opening situated in the circumference wall of the pump body 22. The diameter of the device inlet 12 ranges from 6 to 14 mm, preferably 10 mm. The diameter of the chamber outlet 28 ranges from 6 to 16 mm, preferably 11 mm. The chamber outlet 28 of the compression chamber 24 is connected to the outlet conduct 14 of the device 10.

Once the device 10 is implanted in the patient's heart 100, the device inlet 12 opens into the right atrium 102 of the patient's heart 100 and the outlet conduct 14 opens into the right pulmonary artery 106 of the patient's heart 100. As already mentioned, the blood is therefore pumped from the right atrium 102 or the vena cava 101 into the right pulmonary artery 106 through the first membrane Mi of the patient's heart 100. (see FIGS. 2C, 3A, and 3B).

As can be seen on FIGS. 3A and 3B, the outlet conduct 14 is, contrary to the pump 16, a flexible cylinder which is configured to adapt to the patient's heart 100 morphology. In some embodiments, the outlet conduct 14 can be considered as a meshed stent displaying or other flexible tube materials, preferably partially, with a non-coated surface. Since the outlet conduct 14 is made of flexible braided stent or other flexible tube materials, it can fold and unfold, thus enabling an easy introduction and it can further, once implanted, adapt the patient's heart shape depending on the position of the pump body 22 inside the patient's heart 100. This flexibility further offers the possibility to vary the angle between the pump body 22 and the outlet conduct 14 thus improving the adaptation of the device 10 to the natural shape of the patient's heart 100. The device 10 thus display a general Y shape, or a general T shape, depending on the respective size of the compression chamber 24 with regards to the motor 25 and the position of the chamber outlet 28 on the circumference of the compression chamber 24. The diameter D_{c} of the outlet conduct ranges from 6 to 16 mm, preferably 14 mm.

As can be seen on FIGS. 2A, 2B and 2C, the first support element 20 is configured to be secured to the first membrane M₁ separating the right atrium 102 from the pulmonary artery 106 of the patient's heart 100 by passing through said first membrane M₁.

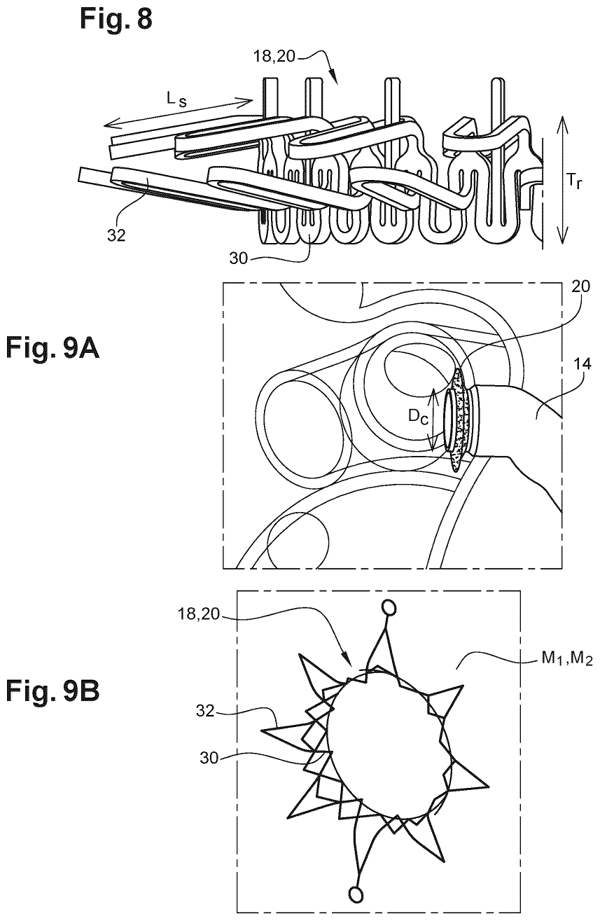

The first support element 20 is configured to tightly cooperate with the outlet conduct 14, more particularly the downflow extremity of the outlet conduct 14 (see FIG. 9A), in order to immobilize the pump 16.

As can be seen on FIGS. 2A, 2B and 2C, in the embodiments in which it is present, the second support element 18 is configured to be secured the second membrane M₂ separating the left atrium 108 from the right atrium 102 of the patient's heart 100 by passing through said second membrane M₂.

As already mentioned, the second support element 18 is configured to cooperate with the pump body 22 in order to immobilize the pump 16 inside the patient's heart 100 (see FIGS. 2C or 6). In those embodiments, the rotor of the pump 16 is surrounded by one extremity 222 of the pump body 22, and the pump 16 is configured to be anchored to the patient's heart 100 at the extremity 222 of the pump body 22 surrounding the rotor. In some embodiments, as can be seen on FIG. 6, the pump 16 is configured to be anchored to the patient's heart 100 by means of a connection element connecting the extremity 222 of the pump body 22 surrounding the rotor to the patient's heart 100. More precisely, the second support element 18 and the pump body 22 cooperate by means of the motor cable 250 connected to the motor 25 at the second extremity 222 of the pump body 22. The motor cable 250 is thus secured to the pump body 22 and to the second support element 18, assuring the function of a connection element and ensuring the pump 16 immobilization.

The first, and when present second, support elements 20, 18 presents sensibly the same structure and shape. Generally speaking, each of the support elements 18, 20 is a cylindrical stent alike element being at least partially made of mashed material. Each first and second support element 18, 20 comprises a central ring 30 designed to be inserted inside a hole created inside a membrane of the patient's heart 100. The diameter Dr of the central ring 30 ranges from 6 to 12 mm and adapts to the diameter of the hole in the membrane. The thickness Tr of the central ring 30 ranges from 1 to 5 mm. The central ring 30 is thus designed to cooperate by friction with the internal walls of the hole (see FIG. 9B). In the embodiment depicted on FIG. 7, the first side of the central ring 30 corresponds to the first extremity of the support elements 18, 20 and the second side of the central ring 30 corresponds to the second extremity of the support elements 18, 20. In order to enable a safe cooperation with the membranes M₁, M₂, each support element 18, 20 comprises two expandable flanges or discs, one on each extremity. The first expandable flange extends from the first extremity of the support element 18, 20 and the second expandable flange extends from the second extremity of the support element 18, 20. The first expandable flange is configured to be located on a first side of the first or second membrane M₁, M₂ and the second expandable flange is configured to be located on a second side of the first or second membrane M₁, M₂. More particularly as can be seen on FIG. 7, the central ring 30 carries on each side, at least one radial strand 32 extending radially outwards the central ring 30. In some embodiment, the central ring 30 carries three radial strands 32 on each side. In the embodiment illustrated on FIG. 7, the central ring 30 carries about twenty radial strands 32 on each side. In the embodiment illustrated on FIG. 9B, the central ring 30 carries 8 radial strands 32 on each side. The length Lₛ of each radial strand ranges from 2 to 10 mm, preferably 4 mm. In the embodiments depicted on FIGS. 7 and 9B, each radial strand is a double strand displaying a general U shape, the free ends of the U being secured to the central ring 30. The radial strands 32 are designed to cooperate, by friction, with the corresponding surface of the membrane surrounding the hole created in the membrane of the patient's heart 100 (see FIG. 9B). The membrane is thus pinched between the radial strand(s) 32 of each side of the central ring 30, on both sides of the crated hole. As the central ring 30 is expendable, the radial strand(s) 32 on each side of the central ring 30 form(s), on each side of the central ring 30 an expandable flange, the expanded configuration of each support element 18, 20 thus enabling the pinching of the first or second membrane M₁, M₂, between the two flanges, or discs. More generally speaking, each support element 18, 20 thus encages the internal walls and the edges of the created hole, and the support element 18, 20 remains thus safely inside the created hole while maintaining said crated hole open. Each support element 20, 18 is configured to resist the pressure difference of the left and the right atrium which is about approximatively 20 mmHg.

The meshed structure of each support element 18, 20 enables a connection with the pump body 22 or the outlet conduct 14. The meshed structure of each support element 18, 20 ensures that they are deployable from a retracted configuration to an expanded configuration. The retracted configuration enables each support element 18, 20 to be safely introduced through the first or second membrane M₁, M₂ of the patient's heart 100, and the expanded configuration enables each support element 18, 20 to stay in place inside the first or second membrane M₁, M₂.

The connection between the first support element 20 and the outlet conduct 14 is configured to be tight, in order to ensure that the blood flow exiting the outlet conduct enters the right pulmonary artery 106 and does not flow back inside the right atrium 102. As the diameter Dₒ of the outlet conduct 14 is larger or equal than the diameter Dᵣ of the central ring 30 of the first support element 20, when the outlet conduct unfolds, it pushes against the internal surface of the central ring 30 and generates a tight connection.

In order to inform the patient about the state of the device 10, the right assisting device 10 is part of a right assisting kit comprising a right assisting device 10 connected to a control unit 34 (see FIGS. 3A and 3B). The control unit 34 comprises a screen, or any kind of interface, which enables to communicate relevant information to the patient or a doctor, for example.

### Implantation Method

In order to implant the right assist device 10 into a patient's heart 100, a implantation method is carried out according to the following steps:
transpiercing the second membrane M₂ with a needle, guidewire or other suitable tools,
transpiercing the first membrane M₁ with a needle or any suitable tool,
introducing, when present, the second support element 18 by means of a catheter inside the hole of the second membrane M₂,
releasing and securing, when present, the second support element 18 inside the first membrane M₂,
introducing the first support element 20 by means of a catheter inside the hole of the second membrane M₁,
releasing and securing the first support element 20 inside the first membrane M₁,
introducing, by means of a catheter, the pump body 22 and the already attached outlet conduct 14 through the second support element 18 inside the right atrium 102 of the patient's heart,
releasing the pump body 22 and the already attached outlet conduct inside the right atrium 102 of the patient's heart 100,
securing the free extremity of the outlet conduct 14 to the first support element 20,
introducing, by means of a catheter, the motor 25 through the second support element 18 inside the right atrium 102 of the patient's heart 100,
pushing the motor 25 inside the second extremity 222 of the pump body 22,
locking the motor 25 inside the pump body 22.

Depending on the embodiment, either the second extremity 222 is directly secured to the second support element 18, or the motor cable 250 already connected to the motor 25 is caught with a lasso, pulled till the tension is adjusted and secured to the second support element 18 in order to immobilize the pump body 22 to the patient's heart 100.

1-16. (canceled)

17. A right cardiac assisting device configured to be percutaneously implanted inside a patient's heart, said assisting device comprising:
an inlet,
an outlet conduct,
a rotary pump comprising a pump body surrounding a rotor, said rotary pump connecting the inlet to the outlet conduct, and being designed to be located inside the right atrium or vena cava of the patient,
wherein
the assisting device further comprises at least one support element, the at least one support element is configured to be secured to a first membrane of the patient's heart by passing through said first membrane, the first membrane separating the right atrium or the vena cava from the pulmonary artery of the patient's heart by passing through said first membrane,
the at least one support element is configured to tightly cooperate with the outlet conduct in order to immobilize the pump, the inlet and the outlet conducts inside the patient's heart or vena cava and enable the patient's blood flow to be driven from the inlet to the outlet conduct through the first membrane of the patient's heart.

18. The right cardiac assisting device according to claim 17, wherein the assisting device further comprises a second support element configured to be secured to a second membrane of the patient's heart by passing through said second membrane, said second support element being configured to cooperate directly or indirectly with the pump body.

19. The right cardiac assisting device according to claim 18, wherein the first membrane is the wall of the superior or inferior vena cava.

20. The right cardiac assisting device according to claim 19, wherein the second membrane is the membrane separating the left atrium from the right atrium of the patient's heart.

21. The right cardiac assisting device according to claim 17, wherein the pump is configured to be anchored to the patient's heart or the vena cava at one extremity of the pump body.

22. The right cardiac assisting device according to claim 21, wherein the rotor of the pump is surrounded by one extremity of the pump body, the pump being configured to be anchored to the patient's heart at the extremity of the pump body surrounding the rotor.

23. The right cardiac assisting device according to claim 22, wherein the pump is configured to be anchored to the patient's heart directly by the extremity of the pump body surrounding the rotor.

24. The right cardiac assisting device according to claim 23, wherein the pump is configured to be anchored to the patient's heart by means of a connection element connecting the extremity of the pump body surrounding the rotor to the patient's heart.

25. The right cardiac assisting device according to claim 17, wherein the device displays a general Y shape or T shape.

26. The right cardiac assisting device according to claim 17, wherein each support elements is deployable from a retracted configuration to an expanded configuration, the retracted configuration enabling each support element to be safely introduced through the first or second membrane of the patient's heart, and the expanded configuration enabling each support element to stay in place inside the first or second membrane.

27. The right cardiac assisting device according to claim 26, wherein each support element comprises two expandable flanges, the expanded configuration of each support element enabling pinching of the first or second membrane between the two flanges.

28. The right cardiac assisting device according to claim 27, wherein a first expandable flange extends from a first extremity of the support element and a second expandable flange extends from a second extremity of the support element, the first expandable flange being configured to be located on a first side of the first or second membrane and the second expandable flange being configured to be located on a second side of the first or second membrane.

29. The right cardiac assisting device according to claim 17, wherein the rotor of the rotary pump is part of a motor designed to be pushed inside the pump body in order to snap the motor and the pump body together.

30. The right cardiac assisting device according to claim 17, wherein the pump body comprises a compression chamber configured to surround an impeller connected to the rotor.

31. A right cardiac assisting kit comprising:
the right cardiac assisting device according to claim 17,
a control unit for controlling the rotary pump of the right cardiac assisting device, and
a power supply for supplying power to the rotary pump.

32. An implantation method for the right cardiac assisting device according to claim 17, wherein the method includes following steps:
transpiercing a first and second membrane of a patient's heart,
securing the first support element of the right cardiac assisting device inside the first membrane,
introducing, the pump body and the outlet conduct of the right cardiac assisting device through the first support element inside the right atrium of the patient's heart,
releasing the pump body and the outlet conduct inside the right atrium,
securing the outlet conduct to the first support element, and
locking the motor inside the pump body.
